(19) United States
(12) Patent Application Publication
(10) Pub. No.: US 2024/0228799 A1
(43) Pub. Date: Jul. 11, 2024

(54) NON-HAZARDOUS MONOMERS AS REACTIVE DILUENTS FOR RESINS

(71) Applicant: ELANTAS EUROPE GMBH, Hamburg (DE)

(72) Inventors: Keiza Fernandes, Hamburg (DE); Sascha Tödter-König; Simon Rost, Büchen (DE)

(21) Appl. No.: 18/554,648

(22) PCT Filed: May 9, 2022
(86) PCT No.: PCT/EP2022/062396

§ 371 Date: Oct. 10, 2023

(30) Foreign Application Priority Data
May 10, 2021 (EP) 21173128.6

(51) Int. Cl. C09D 7/20; C07C 69/80; C08F 283/01; C09D 167/06

(52) U.S. Cl. CPC C09D 7/20; C07C 69/80; C08F 283/01; C09D 167/06

(57) ABSTRACT

The present invention relates to a compound as a reactive diluent for resin compositions as well as a resin composition comprising the same for coating an article and a resin obtained by curing said resin composition.

### TECHNICAL FIELD

The present invention relates to a compound as a reactive diluent for resins as well as a resin composition comprising the same for coating an article and a compound comprising said resin composition.

### TECHNICAL BACKGROUND

Resin compositions for impregnation, coating and sealing of electrical components, like windings of electrical motors or transformers, cables or similar, are conventionally processed by methods known in field of electrical engineering, like dip coating, optionally under elevated temperature with a subsequent UV or heat induced curing step, drip coating, dip rolling, flooding and potting techniques, optionally with an additional application of vacuum or pressure.

The purpose of impregnation, coating and sealing of electrical components is a mechanical stabilization of windings of electrical motors or transformers and their protection from harmful external influences like dust deposition, collector abrasion, salt, humidity or solvent. This prevents mechanical damage during the use of these electrical components and results in an increased lifetime.

Suitable resin compositions for impregnation, coating and sealing of electrical components are conventionally based on unsaturated polyesters, alkyds, epoxies, silicones or mixtures of it diluted in unsaturated acrylic, vinylic or allylic monomers. These unsaturated monomers act as non-polar solvents for the resin and limit the molecular weight of the resin polymer.

The dilution of a resin with unsaturated acrylic, vinylic or allylic monomers is needed to reduce the viscosity of the resin composition. A low viscosity of the resin composition of less than 20.000 mPa·s at 23° C. is mandatory for the use of cost-effective processing methods like trickling, dip coating, roll dipping or hot dipping to achieve a uniform thickness of the resin coating. A low viscosity further results in an increased diffusion of the resin monomers, which slows down the gelation of the resin composition and allows for a more complete reaction of the single components. Consequently, the mechanical properties of the cured resin, like hardness and tensile strength, can be improved substantially.

Examples for conventionally used reactive diluents for unsaturated resins are styrene, acrylates and methacrylates. Those monomers are cheap, easily available, give a favorable viscosity of the resin composition and are simple to polymerize.

Conventional reactive diluents such as styrene, acrylates and methacrylates are easily inflammable and can polymerize spontaneously in an exothermic reaction at ambient temperatures and above. Styrene is known to be carcinogen in contact with eyes and/or skin and due to inhalation or ingestion. It is toxic, especially to ears and eyes of humans, it irritates the respiratory tract and its mutagenic properties are suspected to influence male and female reproduction. Also acrylic acid and methacrylic acid are known to be harmful in contact with eyes and/or skin and due to inhalation or ingestion. Furthermore, styrene, acrylates and methacrylates are classified as hazardous air pollutants. Especially styrene has a high vapor pressure and is a so-called volatile organic compound (VOC). For these reasons, complex and expensive extractor and filter systems are required to protect the people working with these resin compositions from harm. Further, special arrangements for transportation of resin compositions containing these hazardous reactive diluents must be made.

Moreover, conventional reactive diluents are not reacting completely with the polymeric resin, therefore VOC labeled components can evaporate from cured resin coatings, especially due to warming of an electrical component in use and be harmful for customers. Additionally, unreacted monomers can cause further curing of the resin coatings, resulting in undesired hardness or even brittleness of the coating.

For these reasons, reactive diluents, which are less volatile and not harmful, are needed which at the same time provide low viscosity and/or being suitable for impregnation, coating and sealing of electrical components, and upon curing, provide sufficient mechanical and thermal stability for the desired applications.

In WO2018/134291 a solvent-based antifouling composition is described based on a binder made up of multiple monomers. At least one monomer is a polysiloxane unit, the other monomers need to be capable of reacting with the polysiloxane unit through addition polymerization to form ester linkages that are capable of hydrolysis over time in seawater. One of the many other monomers that are mentioned is a di-allyl monomer.

### DETAILED DESCRIPTION OF THE INVENTION

The present invention relates to a compound according to formula

H₂C═CH—Z—Yₐ—O—C(═O)—R—(C(═O)O—Yₐ—Z—CH═CH₂)_{b} (I),
wherein
R is a C₆-C₁₀ aryl, C₅-C₁₀ cycloalkyl, C₃-C₇ heteroaryl, C₃-C₇ heterocyclyl or C₁-C₁₀ alkyl group, optionally substituted with at least one alcohol or amine functional group,
Y is a glycol repeating unit,
Z is a (CH₂) group or a covalent single bond,
a is an integer from 2 to 10, and
b is an integer from 0 to 3,
with the proviso that if Z is a (CH₂) group, b is 1, and R is not substituted with alcohol or amine functional groups, then R is substituted in the 1 and 3 position.

R may be preferably a C₆-C₁₀ aryl, C₃-C₇ heteroaryl or C₁-C₁₀ alkyl group, preferably a C₆-C₁₀ aryl or a C₁-C₁₀ alkyl group, more preferably a phenyl group or a C₁-C₃ alkyl group, optionally substituted with an alcohol functional group.

C₆-C₁₀ aryl group may herein be understood to be an aromatic group like a phenyl, 1-naphthyl or 2-naphthyl group, preferably a phenyl group. If R is C₆ aryl (phenyl), a substitution in the 1 and 3 position means that the phenyl is meta substituted, like in isophathalic acid.

C₃-C₇ heteroaryl group may herein be understood to be an aromatic group containing one heteroatom being part of the organic backbone. The heteroatom may be selected from the group of Sulfur, Oxygen and Nitrogen, preferably from Oxygen. The C₃-C₇ heteroaryl group may preferably be a thienyl, furyl, pyrrolyl or pyridyl group, preferably a furyl group.

C₁-C₁₀ alkyl group may herein be understood to be a linear or branched C₁-C₁₀ alkyl group. The C₁-C₁₀ alkyl group may preferably be a methyl, ethyl, propyl, butyl, pentyl, hexyl, heptyl, octyl, nonyl or decyl group or structural isomers of it, preferably a methyl, ethyl or propyl group or a structural isomer of it, more preferably a methyl group.

C₅-C₁₀ cycloalkyl group may herein be understood to be a cycloalkyl group having a ring size of 5 to 10 carbon atoms. The C₅-C₁₀ cycloalkyl group may preferably be a C₅ or C₆ cycloalkyl group.

R may optionally be substituted with at least one alcohol or amine functional group. Preferably, R may optionally be substituted with one to three alcohol or amine, preferably alcohol, functional groups, preferably with one alcohol or amine, preferably alcohol, functional group.

The optionally substituted R may be a methanolyl, ethanolyl, 1-propanolyl, 2-propanolyl, 1-butanolyl, 2-butanolyl, 1-pentanolyl, 2-pentanolyl, 3-pentanolyl, 1-hexanolyl, 2-hexanolyl, 3-hexanolyl, 1-heptanolyl, 2-heptanolyl, 3-heptanolyl, 4-heptanolyl, 1-octanolyl, 2-octanolyl, 3-octanolyl, 4-octanolyl, 1-nonanolyl, 2-nonanolyl, 3-nonanolyl, 4-nonanolyl, 5-nonanolyl, 1-decanolyl, 2-decanolyl, 3-decanolyl, 4-decanolyl, 5-decanolyl, cyclopentanolyl or cyclohexanolyl group, preferably an ethanolyl, 1-propanolyl, 2-propanolyl, 1-butanolyl, 2-butanolyl group, more preferably a 2-propanolyl group.

The glycol repeating unit Y may herein be understood to be a linear oligomer derived from a condensation reaction of glycol monomers. The glycol repeating unit Y may be independently selected from the groups of ethylene glycol or propylene glycol or combinations thereof, preferably propylene glycol.

The group Z may be a (CH₂) or a covalent single bond. A (CH₂) group may herein be understood to be a methylene group being connected to two additional substituents. The group Z may preferably be a (CH₂) group.

The integer a may be an integer from 2 to 10, preferably from 2 to 6 and even more preferably from 3 to 4.

The integer b may be an integer from 0 to 3, preferably from 1 to 2 and even more preferably 1.

The compound of formula (I) may preferably be di(3,7,11,15-tetraoxaoctadec-17-en-1-yl) isophthalate.

It was found that the compound of formula (I), in particular di(3,7,11,15-tetraoxaoctadec-17-en-1-yl) isophthalate, is non-flammable and has no associated general health hazards (GHS 07) and is also non-hazardous for internal organs (GHS 08).

The present invention also relates to the use of a compound according to formula (I) as described herein-above as a reactive diluent for resin compositions.

A reactive diluent may facilitate the polymerization of an unsaturated resin composition containing such a reactive diluent. A reactive diluent may be used for the preparation of resins, preferably of unsaturated resins.

A reactive diluent may act as non-polar solvent for other components of a resin composition and/or lower the viscosity of an unsaturated resin composition containing such a reactive diluent. The viscosity of an unsaturated resin composition, preferably containing a reactive diluent, may be below 20.000 mPa·s, preferably below 16.000 mPa·s, more preferably below 14.000 mPa·s and even more preferably below 10.000 mPa·s, the viscosity being measured at 23° C. The viscosity of an unsaturated resin composition, preferably containing a reactive diluent, may be at least 100 mPa·s, preferably at least 500 mPa·s at 23° C. The viscosity of an unsaturated resin composition, preferably containing a reactive diluent, may be in the range of 20.000 mPa·s to 100 mPa·s, preferably in the range of 16.000 mPa·s to 500 mPa·s, the viscosity being measured at 23° C.

The reactive diluent may further increase the thermal stability of the cured resin containing such a reactive diluent.

The present invention also relates to a resin composition comprising a compound as described herein-above, an unsaturated base resin, optionally, a further base resin and a curing agent.

A resin composition may herein be understood to be an uncured polymeric composition, which can react to a resin upon curing. Curing may herein be understood to be a chemical process, which may be a polymerization reaction, wherein single monomers or oligomers react with each other to form a tridimensional polymeric network.

The resin composition may preferably be an unsaturated resin composition, preferably an unsaturated polyester resin composition.

An unsaturated resin composition may herein be understood to be a polymeric composition containing unsaturated bonds, which can react with unsaturated bonds of other components of an unsaturated resin composition, preferably with a reactive diluent. This may result in an increase of cross-linking reactions within the resin composition during the curing step and therefore may improve the properties of the cured resin.

An unsaturated resin composition may herein be an unsaturated polyester resin composition.

An unsaturated polyester resin composition may herein be understood to be a polymeric composition, which may contain a multivalent alcohol, preferably a divalent alcohol such as a glycol, and a dicarboxylic acid, such as maleic acid, fumaric acid, trimellitic acid or a dimerized fatty acid, preferably maleic acid, or any anhydride of it, like maleic anhydride or trimellitic anhydride, preferably maleic anhydride.

The resin composition comprises an unsaturated base resin. An unsaturated base resin may herein be understood to be a composition comprising unsaturated monomers or oligomers that are suitable to react with each other upon curing to form an unsaturated resin.

The unsaturated base resin may be a polyester base resin, which may contain a multivalent alcohol, preferably a divalent alcohol like a glycol, and a dicarboxylic acid like maleic acid, fumaric acid, trimellitic acid or a dimerized fatty acid, preferably maleic acid, or any anhydride of it, like maleic anhydride or trimellitic anhydride, preferably maleic anhydride.

The resin composition may optionally comprise a further base resin.

The further base resin may comprise a vinyl ester base resin, an acrylic base resin, a silicone base resin or mixtures thereof.

The vinyl ester base resin may contain an epoxy resin and acrylic acid or methacrylic acid. The acrylic base resin may contain acrylic acid, methacrylic acid, methyl acrylate and/or methyl methacrylate. The silicone base resin may contain polymerized siloxanes like polydimethylsiloxanes or oligosiloxanes.

The unsaturated base resin may further, but not necessarily, comprise a monovalent alcohol, preferably N-(2-hydroxyethyl)phthalimide, and/or a trivalent alcohol, preferably tris (2-hydroxyethyl) isocyanurate.

The resin composition further comprises a curing agent.

A curing agent may herein be understood to be a chemical compound initiating a polymerization reaction, wherein single monomers or oligomers react with each other to form a tridimensional polymeric network. The polymerization reaction may herein be a free-radical polymerization, a cationic polymerization or an anionic polymerization, preferably a free-radical polymerization.

A free-radical polymerization may be initiated by a chemical compound comprising a peroxide functionality, which can produce free-radical species under mild conditions and promote free-radical reactions.

The curing agent may be tert-butyl peroxybenzoate (TBPB), 1,1-di(tert-butylperoxy)-3,5,5-trimethylcyclohexane or tert-butyl cumyl peroxide or a mixture of it, preferably tert-butyl peroxybenzoate (TBPB).

A low reaction enthalpy of the resin composition comprising a curing agent is preferred as it facilitates a uniform curing reaction and/or a long shelf life. The reaction enthalpy of the resin composition comprising a curing agent may be below 800 J g⁻¹, preferably below 600 J g⁻¹, more preferably below 400 J g⁻¹. The reaction enthalpy of the reactive diluent containing 2% by weight of the curing agent, preferably TBPB, may be below 600 J g⁻¹, preferably below 300 J g⁻¹, more preferably below 150 J g⁻¹. The reaction enthalpy can be measured in a DSC-measurement.

The present invention also relates to a resin obtainable by curing a resin composition as described herein-above.

A resin may herein be understood to be a cured resin composition. Curing may herein be understood to be a chemical process, which may be a polymerization reaction, wherein single monomers or oligomers react with each other to form a tridimensional polymeric network.

The resin may preferably be an unsaturated polyester resin composition, preferably alkyl ester resin composition, vinyl ester resin composition or acrylic resin composition.

An unsaturated resin may herein be understood to be a polymeric composition containing unsaturated bonds, which reacted with unsaturated bonds of other components of an unsaturated resin composition, preferably with a reactive diluent. This may result in an increased cross-linking within the resin and therefore may improve the properties of the resin.

An unsaturated resin may herein be an unsaturated polyester resin.

An unsaturated polyester resin may herein be understood to be a polymeric composition, which may be derived from a condensation reaction of a multivalent alcohol, preferably a divalent alcohol like a glycol, and a dicarboxylic acid like maleic acid, fumaric acid, trimellitic acid or a dimerized fatty acid, preferably maleic acid, or any anhydride of it, like maleic anhydride or trimellitic anhydride, preferably maleic anhydride.

The unsaturated resin may preferably be derived from an unsaturated polyester base resin, which may contain a multivalent alcohol, preferably a divalent alcohol like a glycol, and a dicarboxylic acid like maleic acid, fumaric acid, trimellitic acid or a dimerized fatty acid, preferably maleic acid, or any anhydride of it, like maleic anhydride or trimellitic anhydride, preferably maleic anhydride, a vinyl ester base resin, which may contain an epoxy resin and acrylic acid or methacrylic acid, or an acrylic base resin, which may contain acrylic acid, methacrylic acid, methyl acrylate and/or methyl methacrylate, or a silicone base resin, which may contain polymerized siloxanes like polydimethylsiloxanes or oligosiloxanes or mixtures therefrom, more preferably be derived from an unsaturated polyester base resin.

The present invention also relates to the use of a resin composition as described herein-above for coating an article, preferably an electrical component.

A person skilled in the art is aware of methods of coating of an article. Non-limiting examples of coating comprise spray coating, roll-to-roll coating, dip coating, spin coating, trickling, roll dipping or hot dipping and the like. Coating is herein understood as a partial coating, such as at least 50%, preferably at least 60% coating, more preferably at least 80% coating of the surface of the article or a complete coating, i.e. 100% of the surface, of the article.

An article may be an electrical component, such as windings of electrical motors or transformers, cables or the like.

The resin as described herein-above may be used for insulating electrical components. It was found that the reactive diluent in accordance with the present invention in a composition for an insulating electrical component with an unsaturated resin, in particular an unsaturated polyester resin, results in a cured composition with a higher thermal index that known insulating electrical components based on an unsaturated polyester resin. Also the resistance to automobile oils of a composition for insulating electrical components comprising the reactive diluent according to the present invention is better than known compositions for insulating electrical components.

In the following, several examples are given to illustrate the present invention, however, they are not meant to limit the scope of the invention. Other embodiments of the invention may readily be prepared on the basis of the general teaching herein and the following examples.

### Measurement Methods

### Viscosity

The viscosity of the various materials was measured in accordance with DIN 53019 using a Physica Rheometer Z3.

The measurements are performed at 23° C. and shear rate of 12,9 s⁻¹ for higher viscous materials or fluid materials having an (expected) viscosity >1000 mPa·s and at a shear rate of 90 s⁻¹ for more lower materials having an expected viscosity of ≤ 1000 mPa·s. Prior to testing, the sample should as free as possible of air bubbles. The measured value is specified in milli Pascal seconds (mPa·s).

### Temperature Index

The temperature index was measured in accordance with IEC 60455-2 item 6.5.10

### Resistance to Automobile Oils

The resistance to automobile oils was measured in accordance with IEC 60455-2 item 6.5.2 using different grades of Fuchs oil. In this test a metal plate is coated with a resin, the resin is cured and the coated metal plate is weighed. Thereafter the coated metal plate is immersed in the oil for a specified time. Then, the coated metal plate is removed from the oil and dried and weighed again. An increase of weight means that the coating has absorbed some of the oil probably by swelling, a decrease means the coating has degraded. The smaller the weight change, the more resistant the coating is to the oil.

### EXAMPLES

### Example 1: Preparation of the Diallyl Isophthalate Ester

1 mol of dimethyl isophthalate is reacted with 2.5 moles of a polyglycol allylalcohol with 3.5 repeating units in the presence of 0.2% w/w dibutyltinoxide at 140° C. under nitrogen. During the reaction, formed methanol is constantly removed by distillation at a column temperature of 60-65° C. The transesterification process is continued under constantly increasing temperature up to 180° C. After completing the transesterification process, nitrogen is switched off and vacuum is slowly applied until a pressure of −980 mbar is reached. Methanol and excess polyglycol allylalcohol are removed at a column temperature of 90° C. The final product was analyzed by gas chromatography—mass spectrometry (GC-MS) and nuclear magnetic resonance (NMR) spectroscopy, indicating a product mainly comprising of di(3,7,11,15-tetraoxaoctadec-17-en-1-yl) isophthalate with a yield of 96.9%.

Gel permeation chromatography (GPC) revealed an average molecular weight of 650 Da, which matches the theoretical value of di(3,7,11,15-tetraoxaoctadec-17-en-1-yl) isophthalate.

### Example 2: Properties and Stability of Diallyl Isophthalate Ester

The diallyl isophthalate ester prepared in Example 1 is a stable liquid at room temperature (25° C.) with a viscosity of 50 mPa·s at 23° C. and a vapor pressure below 0.1 hPa and is therefore considered to be a non-VOC solvent.

The diallyl isophthalate ester prepared in Example 1 was stored for one year at room temperature (25° C.) to check its stability. After mixing the diallyl isophthalate ester with 2% by weight of a curing agent (tert-butyl peroxybenzoate (TBPB)), the reaction enthalpy of the system remains unchanged compared to freshly prepared diallyl isophthalate ester.

The viscosity of the system is stable and no sedimentation or gelification was observed. Freshly prepared diallyl isophthalate ester exhibits a viscosity of 55 mPa·s at 23° C., while the stored diallyl isophthalate ester exhibits a viscosity of 50 mPa·s at 23° C. after storage.

In the following experiments, different base resins are mixed with a compound according to invention used as reactive diluent, together with an inhibitor and an initiator. Comparative examples are given by using alternative, conventionally used methacrylic or acrylic reactive diluent.

### Example 3

### Unsaturated Base Resin 1:

N-(2-Hydroxyethyl) phthalimide, maleic anhydride, dicyclopentadiene (DCPD), triglycol and propandiol.

### Test Composition 1 (According to the Invention)

71.98% unsaturated base resin 1 is mixed with 25% diallyl isophthalate ester, 0.02% inhibitor and 3% initiator. The resulting resin composition has a viscosity of 10000 to 14000 mPa·s at 23° C. and a gel time below 15 min at 120° C.

### Comparative Composition 1 (Methacrylic Reactive Diluent)

69.2% unsaturated base resin 1 is mixed with 26.2% methacrylic reactive diluent, 0.05% inhibitor, 4.5% initiator. The resulting resin composition has a viscosity of 18000 to 22000 mPa·s at 23° C. and a gel time below 10 min at 120° C.

### Example 4

### Unsaturated Base Resin 2

N-(2-Hydroxyethyl) phthalimide, maleic anhydride, tris (2-hydroxyethyl) isocyanurate (THEIC), diethylene glycol and propanediol.

### Test Composition 2 (According to the Invention)

58% unsaturated base resin 2 is mixed with 40% diallyl isophthalate ester, 0.15% inhibitor (10% p-benzoquinone solution and di-tert-butyl-p-kresol), 3% curing agent (TBPB), 1,1-di(tert-butylperoxy)-3,5,5-trimethylcyclohexane and tert-butyl cumyl peroxide and 0.52% additive (epoxidized soya bean oil as plasticizer and a polyacrylate base as levelling agent). The resulting resin composition has a viscosity of 7000 to 11000 mPa·s at 23° C. and a gel time below 10 min at 120° C.

### Comparative Composition 2 (Acrylic Reactive Diluent)

60% unsaturated base resin 2 is mixed with 39% acrylate monomer mix (7.5% tricyclodecanedimethanol dimethacrylate, 10% poly (ethylene glycol) dimethacrylat (PEGDMA) and 21.5% tri(ethyleneglycol) dimethacrylat (TEGDMA)), 0.2% inhibitor (10% p-benzoquinone solution), 0.99% curing agent (TBPB) and 0.53% additive (epoxidized soya bean oil as plasticizer and a polyacrylate base as levelling agent). The resulting resin composition has a viscosity of 6000 to 9000 mPa·s at 23° C. and a gel time below 10 min at 120° C.

### Example 5

### Unsaturated Base Resin 3:

Trimellitic anhydride, maleic anhydride, N-(2-Hydroxyethyl) phthalimide and neopentyl glycol.

### Test Composition 3 (According to the Invention)

52.5% unsaturated base resin 3 is mixed with 43% diallyl isophthalate ester, 0.26% inhibitor (10% p-benzoquinone solution and di-tert-butyl-p-kresol), 1.5% curing agent and 1.9% additive (epoxidized soya bean oil as plasticizer). The resulting resin composition has a viscosity of 8000 to 12000 mPa·s at 23° C. and a gel time below 12 min at 120° C.

### Comparative Composition 3 (Acrylic Reactive Diluent)

48% unsaturated base resin 3 is mixed with 50% acrylate monomer mix (6% PEGDMA and 44% TEGDMA), 0.12% inhibitor (10% p-benzoquinone solution and di-tert-butyl-p-kresol), 2.1% curing agent, 0.012% accelerator (manganese octoate) and 2.1% additive (epoxidized soya bean oil as plasticizer). The resulting resin composition has a viscosity of 1600 to 2000 mPa·s at 23° C. and a gel time of 3 to 7 min at 120° C.

In the following comparative experiments, styrene is used as reactive diluent and used in combination with different base resins.

### Example 6

### Comparative Composition 4 (Styrene as Reactive Diluent)

Comparative test composition 3 is a two-component system wherein component A comprises 54.6% unsaturated base resin 3, 43.14% styrene, 0.27% inhibitor (10% p-benzoquinone solution and di-tert-butyl-p-kresol) and 2% curing agent (TBPB) and wherein component B comprises 54.6% unsaturated base resin 3, 38.2% styrene, 0.25% inhibitor (10% p-benzoquinone solution and di-tert-butyl-p-kresol) and 2.15% acetylacetonat. The resulting resin composition has a viscosity of 115 to 135 mPa·s at 23° C. and gel time of 5 to 7 min at 100° C.

### Example 7

### Unsaturated Base Resin 4:

N-(2-Hydroxyethyl) phthalimide, maleic anhydride, THEIC, a dimerized fatty acid and neopentyl glycol.

### Comparative Composition 5 (Styrene as Reactive Diluent)

34.4% unsaturated base resin 3 is mixed with 32.3% unsaturated base resin 4, 33.4% styrene, 0.17% inhibitor (10% p-benzoquinone solution and di-tert-butyl-p-kresol) and 1% curing agent (TBPB). The resulting resin composition has a viscosity of 500 to 540 mPa·s at 23° C. and gel time of 32 to 39 min at 100° C.

### Example 8: Properties of the Resin Compositions

The resin compositions described in Examples 3 to 7 were cured at 120° C. for one hour and for 160° C. for two hours. Afterwards, all resin compositions were tested for their thermal, mechanical, electrical and chemical resistance properties.

TABLE 1
Description of relevant properties - part 1

| | | Test | Comparative | Test | Comparative |
|---|---|---|---|---|---|
| | | composition | composition | composition | composition |
| Property | Unit | 1 | 1 | 2 | 2 |
| Type of reactive diluent | | according to | methacrylic | according to | acrylic |
| | | the invention | | the invention | |
| Reaction enthalpy | J/g | 392 | 319 | 240 | 218 |
| Curing onset temperature | | 125 | 128 | 126 | 130 |
| Curing efficiency | % | 99 | 99 | 99.9 | 99 |
| Emission | % | 3.7 | — | 2.5 | — |
| Electrical Strength at 23° C. | $\frac{kV}{mm}$ | 251 | 171 | 257 | 265 |
| Electrical Strength at 180° C. | $\frac{kV}{mm}$ | 162 | 203 | 175 | 176 |
| Resistance against | | Resistant | Not Resistant | Resistant | Not Resistant |
| ammonia 10% | | | | | |
| Resistance against acetic | | Resistant | Not Resistant | Resistant | Resistant |
| acid 10% | | | | | |
| Solvent vapor Pressure at | hPa | <0.01 | <0.01 | <0.01 | <0.01 |
| 20° C. | | | | | |
| GHS hazard pictograms | | None | None | None | GHS 07: |
| | | | | | Harmful |
| Hazard signal word | | None | None | None | Warning |

TABLE 2
Description of relevant properties - part 2

| | | Test | Comparative | Comparative | |
|---|---|---|---|---|---|
| | | composition | composition | composition | Comparative |
| Property | Unit | 3 | 3 | 4 | composition 5 |
| Type of reactive diluent | | according to | acrylic | styrene | styrene |
| | | the invention | | | |
| Enthalpy | J/g | 240 | 274 | 255 | 248 |
| Onset | ° C. | 128 | 121 | 125 | 126 |
| Curing | % | 99 | 99 | 99 | 99 |
| Emission | % | 3.5 | — | 27 | 22 |
| Electrical Strength at 23° C. | $\frac{kV}{mm}$ | 292 | 186 | 168 | 130 |
| Electrical Strength at 180° C. | $\frac{kV}{mm}$ | 177 | 192 | 139 | — |
| Resistance against | | Resistant | Not Resistant | Resistant | Not Resistant |
| ammonia 10% | | | | | |
| Resistance against | | Resistant | Resistant | Resistant | Partial |
| acetic acid 10% | | | | | |
| Solvent vapor Pressure at | hPa | <0.01 | <0.01 | 7 | 7 |
| 20° C. | | | | | |
| GHS hazard pictograms | | None | GHS 07: | GHS 07: | GHS 07: |
| | | | Harmful | Harmful; 02 | Harmful; 02 |
| | | | | Flammable; | Flammable; |
| | | | | 08 Health | 08 Health |
| | | | | hazard | hazard |
| Hazard signal word | | None | Warning | Danger | Danger |

### Example 9: Thermal Index

The thermal index (TI) using an helical coil was measured in accordance with IEC 60455-2 for some materials. The results are presented in Table 3.

TABLE 3
Results of Thermal index testing

| | | Test | Test | Comparative |
|---|---|---|---|---|
| Property | Unit | composition 1 | composition 2 | composition 1 |
| TI | ° C. | 235 | 228 | 213 |
| helical coil | | | | |

### Example 10: Resistance to Automobile Oils

The resistance to automobile oils was measured in accordance with IEC 60455-2 for some materials. The results are presented in Table 3.

TABLE 4
Results of resistance to automobile oils, measured as weight change

| Type of oil | | Test | Comparative | Comparative |
|---|---|---|---|---|
| (FES) | Unit | composition 1 | composition 2 | composition 3 |
| 215-5091 LV | mg | 3 | 9 | |
| 218-5535 A | mg | 4 | 16 | |
| 219-5698 | mg | 5 | 11.5 | 9 |
| Titan EG 52529 | mg | 3 | 9 | 8 |

1. A compound according to formula
H₂C═CH—Z—Yₐ—O—C(═O)—R—(C(═O)O—Yₐ—Z—CH═CH₂)_{b} (I),
wherein
R is a C₆-C₁₀ aryl, C₅-C₁₀ cycloalkyl, C₃-C₇ heteroaryl, C₃-C₇ heterocyclyl or C₁-C₁₀ alkyl group, optionally substituted with at least one alcohol or amine functional group,
Y is a glycol repeating unit,
Z is independently selected from a (CH₂) group or a covalent single bond,
a is an integer from 2 to 10, and
b is an integer from 0 to 3,
with the proviso that if Z is a (CH₂) group, b is 1, and R is not substituted with alcohol or amine functional groups, then R is substituted in the 1 and 3 position.

2. The compound according to claim 1, wherein R is a C₆-C₁₀ aryl, C₃-C₇ heteroaryl or C₁-C₁₀ alkyl group optionally substituted with an alcohol functional group.

3. The compound according to claim 1, wherein the glycol repeating unit Y is independently selected from the groups of ethylene glycol or propylene glycol.

4. The compound according to claim 1, wherein Z is a (CH₂) group.

5. The compound according to claim 1, wherein a is an integer from 2 to 6.

6. The compound according to claim 1,
wherein b is an integer from 1 to 2.

7. The compound according to claim 1, wherein the compound of formula (I) is di(3,7,11,15-tetraoxaoctadec-17-en-1-yl) isophthalate.

8-9. (canceled)

10. A resin composition comprising
a. the compound as defined in claim 1,
b. an unsaturated base resin
c. optionally a further base resin, and
d. a curing agent.

11. The resin composition according to claim 10, wherein the resin composition comprises an unsaturated polyester resin composition.

12. The resin composition according to claim 10, wherein the unsaturated base resin is a polyester base resin, which is composed from a multivalent alcohol, and a dicarboxylic acid comprising one or more of maleic acid, fumaric acid, trimellitic acid or a dimerized fatty acid, or any combination thereof.

13. A resin obtained by curing the resin composition according to claim 10.

14. An electrical component coated with the resin of claim 10.

15. (canceled)

16. The compound according to claim 2, wherein R is a C₆-C₁₀ aryl or a C₁-C₁₀ alkyl group optionally substituted with an alcohol functional group.

17. The compound according to claim 2, wherein R is a phenyl group or a C₁-C₃ alkyl group optionally substituted with an alcohol functional group.

18. The resin composition according to claim 10, wherein the unsaturated base resin is a polyester base resin composed from a glycol and one or more of maleic acid, maleic anhydride or trimellitic anhydride.

19. The resin composition according to claim 10, wherein the unsaturated base resin comprises an alkyl ester resin composition, a vinyl ester resin composition, or an acrylic resin composition.

20. The resin composition according to claim 10, wherein the glycol repeating unit Y is independently selected from the groups of ethylene glycol or propylene glycol.

21. The resin composition according to claim 10, wherein Z is a (CH₂) group.

22. The resin composition according to claim 10, wherein the compound of formula (I) is di(3,7,11,15-tetraoxaoctadec-17-en-1-yl) isophthalate.
